(19) United States
(12) Patent Application Publication
(10) Pub. No.: US 2016/0349252 A1
(43) Pub. Date: Dec. 1, 2016

(54) SYSTEMS AND METHODS FOR COMBINED VERTICAL/LATERAL FLOW BLOOD SEPARATION TECHNOLOGIES WITH ENABLEMENT OF POINT-OF-CARE COTININE DETECTION WITH EXTENDED RANGE

(71) Applicant: Polymer Technology Systems, Inc., Indianapolis, IN (US)

(72) Inventors: Keith Moskowitz, Indianapolis, IN (US); Christopher Dailey, Indianapolis, IN (US); Charles Xie, Indianapolis, IN (US); Richard Lee, Indianapolis, IN (US)

(21) Appl. No.: 15/167,533

(22) Filed: May 27, 2016

Related U.S. Application Data
(60) Provisional application No. 62/168,597, filed on May 29, 2015.
(60) Provisional application No. 62/170,390, filed on Jun. 3, 2015.

(51) Int. Cl. G01N 33/543; G01N 33/577; G01N 33/94

(52) U.S. Cl. CPC G01N 33/54386; G01N 33/94; G01N 33/54313; G01N 33/577

(57) ABSTRACT

A system for determining a level of cotinine in a sample includes a test strip system configured to receive a sample, the test strip system including a first lateral flow test strip and a second lateral flow test strip, the first and second lateral flow test strips each having an overlapping but non-identical range for cotinine. The system further includes a meter configured to receive the test strip, wherein the meter is configured to read the test strip and detect a level of cotinine.

### CROSS-REFERENCE TO RELATED APPLICATIONS

This Application claims the benefits of Provisional Application No. 62/168,597 filed May 29, 2015, entitled “Systems And Methods For Distinguishing Cotinine From Anabasine In A Point-Of-Care Testing Device,” and Provisional Application No. 62/170,390 filed Jun. 3, 2015, entitled “Systems And Methods For Combined Vertical/Lateral Flow Blood Separation Technologies With Enablement Of Point-Of-Care Cotinine Detection With Extended Range,” the entire disclosures of which are hereby incorporated by reference.

### BACKGROUND

According to the Centers for Disease Control and Prevention, smoking is the leading cause of preventable death in the United States. The first published studies on the harmful effects of smoking on health were retrospective analyses of the smoking habits of patients suffering from lung cancer in 1950. The major harmful effects attributed to smoking include, but are not limited to, heart disease, stroke, chronic obstructed pulmonary disease, and numerous cancers. While initially attributed to primary smoking activities, the harmful effects on the health of an individual extend to those exposed passively to tobacco smoke from the environment. These health consequences of tobacco use substantially increase the cost of healthcare. In 2014, the US Department of Health and Human Services issued a report “Health Consequences of Smoking—50 Years of Progress—A Report of the Surgeon General” estimating the economic costs resulting from lost productivity as a consequence from both early mortality and associated health care costs. Lost productivity across all demographics and disease states for adults 35-79 between the years 2005-2009 was estimated to be $151 billion. Aggregate health care expenditures attributable to cigarette smoking for adults 35 and older in 2012 alone was estimated to be $175.9 billion. Tobacco cessation initiatives have been created by both employer-based health care systems and public health systems to curb these economic losses and improve public health. However, monitoring for adherence to these cessation initiatives often relies on self-reporting. Literature reviews of the effectiveness of self-reporting screening for a wide variety of risk factors, including tobacco use, consistently finds significant under reporting, decreasing opportunities for interventions.

Tobacco exposure determination relies on the detection of substances directly or indirectly associated with tobacco use. Tobacco contains numerous structurally similar alkaloids with the principle alkaloid, nicotine, making up about 95% of the total alkaloid content. Nicotine is the primary addictive substance in tobacco, resulting in strong physical and psychological dependence, making nicotine replacement therapy (NRT) the leading choice in cessation activities as it assists the individual to reduce nicotine intake without exposure to tobacco.

Current tests available for detection of tobacco are carbon monoxide, nicotine, and cotinine in varying matrices, such as urine, blood, breath, and/or saliva. However, plasma nicotine and carbon monoxide have short half-lives that may allow a person to stop smoking for a short time and test as a non-smoker. Cotinine, the major metabolite of nicotine, has been the metabolite of choice as it is the most abundant. It can be measured via a central lab in urine, saliva, or plasma. Point-of-care or near-patient settings currently are limited to qualitative tests from urine and saliva, complicating sampling collection and sample processing.

Objectively detecting exposure to tobacco, eliminating the need for self-reporting, can be achieved by detecting substances directly absorbed by the body from tobacco or the metabolites and/or catabolites of these substances, instead of the more traditional cotinine, nicotine, or carbon monoxide testing. Detectable tobacco alkaloids include nicotine, anabasine, and anatabine, with numerous metabolites, only a few of which possess pharmacokinetics and pharmacokinetic characteristics that are desirable as indicators of tobacco exposure. The primary characteristics indicative of an effective indicator of tobacco exposure are long half-lives and overall abundance of the substance in the applicable matrix (i.e. urine, whole blood, plasma, saliva, etc.).

Thus, there is a need in the art to develop testing methods for the quantitative determination of cotinine from biofluids, including whole blood at the point-of-care and near care environments.

### BRIEF SUMMARY

In one embodiment, a system for determining a level of cotinine in a sample includes a test strip system configured to receive a sample, the test strip system including a first lateral flow strip and a second lateral flow test strip, the first and second lateral flow test strips each having an overlapping but non-identical range for cotinine. The system further includes a meter configured to receive the test strip, wherein the meter is configured to read the test strip and detect a level of cotinine. Optionally, the first and second lateral flow test strips each include microparticles combined with a cotinine antibody. Alternatively, the first and second lateral flow test strips each include antigens to bind with the microparticles combined with a cotinine antibody. Optionally, for the first test strip and second test strip, antibodies only need to recognize cotinine and may be of different origins and/or have been produced using unique immunogens to achieve distinct characteristics such as affinities and avidities towards cotinine. In one configuration, for the first and second test strips, the microparticles combined with respective cotinine antibodies are independently optimal for high sensitivity and dynamic range. The combination of the first and second test strips results in a larger testing range for cotinine using the test strip system. In any embodiment, the specific antibodies employed may be monoclonal or polyclonal in nature. Alternatively, the test strip system includes a red blood cell separation membrane. Optionally, the red blood cell separation membrane is a vertical flow membrane. Alternatively, the test strip system includes a sample pad oriented in line with an opening in a cartridge, the cartridge holding the sample pad, the red blood cell separation membrane, and the first and second lateral flow test strips. Optionally, the test strip system includes a wicking membrane in the cartridge; and the cartridge holding the sample pad, the red blood cell separation membrane, and the wicking membrane forms a stack of membranes in that order, the stack of membranes being approximately in vertical alignment with the opening, and the wicking membrane oriented in contact with the first and second lateral flow test strips in order to provide sample to the lateral flow test strip.

In one embodiment, a system for determining a level of cotinine in a sample includes a test strip system configured to receive a sample and a meter configured to receive the test strip, the meter being configured to read the test strip and detect a level of cotinine. Optionally, the test strip system includes a red blood cell separation membrane, which may include a system of membranes based on lateral or vertical flow membranes. Alternatively, the test strip system includes a lateral flow test strip. In one alternative, the red blood cell separation membrane is a vertical flow membrane. In another alternative, the test strip system includes a sample pad oriented in line with an opening in a cartridge, the cartridge holding the sample pad, the red blood cell separation membrane, and the lateral flow test strip. Optionally, the test strip system includes a wicking membrane in the cartridge. Alternatively, the cartridge holding the sample pad, the red blood cell separation membrane, and the wicking membrane forms a stack of membranes in that order, the stack of membranes being approximately in vertical alignment with the opening, the wicking membrane oriented in contact with the lateral flow test strip in order to provide sample to the lateral flow test strip. Optionally, the lateral flow test strip includes microparticles combined with a cotinine antibody. Alternatively, the test strip includes a first test site, the first test site including compounds, which may be antigens, to bind with the microparticles combined with a cotinine antibody. In one configuration, the microparticles are fluorescent. In another configuration, the microparticles have reflective properties. Optionally, the microparticles have properties that provide for the absorption of light. In another configuration, the meter measures a level of absorption at the first test site to determine the level of cotinine. Optionally, the meter measures a level of reflection at the first test site to determine the level of cotinine.

In another embodiment, a test strip system for determining a level of cotinine in a sample includes a red blood cell separation membrane and a lateral flow test strip, wherein the lateral flow test strip includes microparticles combined with a cotinine antibody. Alternatively, the red blood cell separation membrane is a vertical flow membrane. Optionally, the test strip further includes a sample pad and a cartridge, the sample pad oriented in line with an opening in a cartridge, the cartridge holding the sample pad, the red blood cell separation membrane, and the lateral flow test strip. Optionally, the test strip further includes a wicking membrane in the cartridge. Alternatively, the cartridge holding the sample pad, the red blood cell separation membrane, and the wicking membrane forms a stack of membranes in that order, the stack of membranes being approximately in vertical alignment with the opening, the wicking membrane oriented in contact with the lateral flow test strip in order to provide sample to the lateral flow test strip. Optionally, the test strip includes a first test site, the first test site including compounds to bind with the microparticles combined with a cotinine antibody.

In one embodiment, a method of determining a level of cotinine in a sample includes providing a test strip system configured to receive a sample wherein the test strip system includes microparticles combined with a cotinine antibody and providing a meter configured to receive the test strip wherein the meter is configured to read the test strip and detect a level of cotinine. The method further includes placing a sample on the test strip, laterally flowing the sample on the test strip, and reading the test strip with the meter. Optionally, the test strip system includes a sample pad and a cartridge, the sample pad oriented in line with an opening in a cartridge, the cartridge holding the sample pad, the red blood cell separation membrane, and the lateral flow test strip. Optionally, the test strip system includes a wicking membrane in the cartridge. Alternatively, the cartridge holding the sample pad, the red blood cell separation membrane, and the wicking membrane forms a stack of membranes in that order, the stack of membranes being approximately in vertical alignment with the opening, the wicking membrane oriented in contact with the lateral flow test strip in order to provide sample to the lateral flow test strip. Optionally, the method further includes binding at least a portion of cotinine with microparticles combined with the cotinine antibody and binding at least a portion of the microparticles combined with the cotinine antibody to a first test site. The method of reading the test strip includes detecting at the first test site to determine the level of cotinine.

### BRIEF DESCRIPTION OF THE DRAWINGS

FIG. 1 shows one embodiment of a cartridge for use with a meter for reading a color change;

FIG. 2 shows one embodiment of a schematic for competitive-inhibition, particle-capture immunoassay;

FIG. 3 demonstrates the effect of the included red blood cell (RBC) separation step;

FIG. 4 demonstrates the effect of interference of RBCs of the reflectance measurement;

FIG. 5 shows results of embodiment of red blood cell separation;

FIG. 6 shows an alternative embodiment for a cartridge for detecting cotinine;

FIG. 7a shows a detailed view of one layer of the cartridge of FIG. 6;

FIG. 7b shows a detailed view of one layer of the cartridge of FIG. 6;

FIG. 7c shows a detailed view of one layer of the cartridge of FIG. 6;

FIG. 8 shows a perspective view of the cartridge of FIG. 6;

FIG. 9 shows one embodiment of a graph showing an extended dynamic range for cotinine detection;

FIG. 10 shows an example of cotinine 3 and cotinine 4; and

FIG. 11 shows an alternative embodiment of a cartridge including a red blood cell separation membrane.

### DETAILED DESCRIPTION

Certain terminology is used herein for convenience only and is not to be taken as a limitation on the embodiments of the systems and methods for combined vertical/lateral flow blood separation technologies with enablement of point-of-care cotinine detection with extended range. In the drawings, the same reference letters are employed for designating the same elements throughout the several figures.

Currently, all point-of-care tests for the detection of cotinine, a metabolite of nicotine, are based on oral fluid (saliva) or urine and only provide qualitative or semi-quantitative results. To achieve a quantitative result, the samples must be sent to a central lab for analysis by liquid chromatography-tandem mass spectrometry (LC-MS/MS). These results often can take over a week, substantially limiting the window of opportunity for education and intervention.

In one embodiment, a system is capable of quantifying cotinine from a whole blood sample in a point-of-care setting without the need for sophisticated laboratory equipment. The system includes an on-device red blood cell (RBC) separation component.

In many embodiments, the system includes a lateral flow cotinine assay with a quantitative dynamic range from 25 ng/mL to 200 ng/mL. In some embodiments, the range may be as low as 10 ng/ml. Many embodiments of the system further include an on-device sample processing system capable of providing RBC-depleted samples to lateral flow test strips. The inclusion of such eliminates the need for large complex separation systems in many scenarios.

Prior solutions for measuring cotinine in point-of-care solutions have focused on oral fluid and urine, where the device disclosed here is capable of quantifying cotinine from whole blood sampled from either a finger stick or a venous draw. In addition, the device disclosed here provides a quantitative result without the need for expensive and sophisticated analysis through a central laboratory.

Embodiments of the system physically separate the RBC separation from the lateral flow strips by performing the RBC filtration in a separate plane, substantially limiting the probability of inadvertent contamination of the lateral flow test strip with RBCs. This system to remove the RBCs from sample prior to contact with the test strips requires no additional steps or intervention by the user, substantially increasing the usability and accessibility of the device to the general population. Without this separation, the typical solution of depleting sample of RBCs includes a combination of filtration and capture (through anti-RBC antibodies, lectins, or other RBC capturing agents) that often occur on a separate device using moderately complex equipment or other manual steps that require sample manipulation by the user.

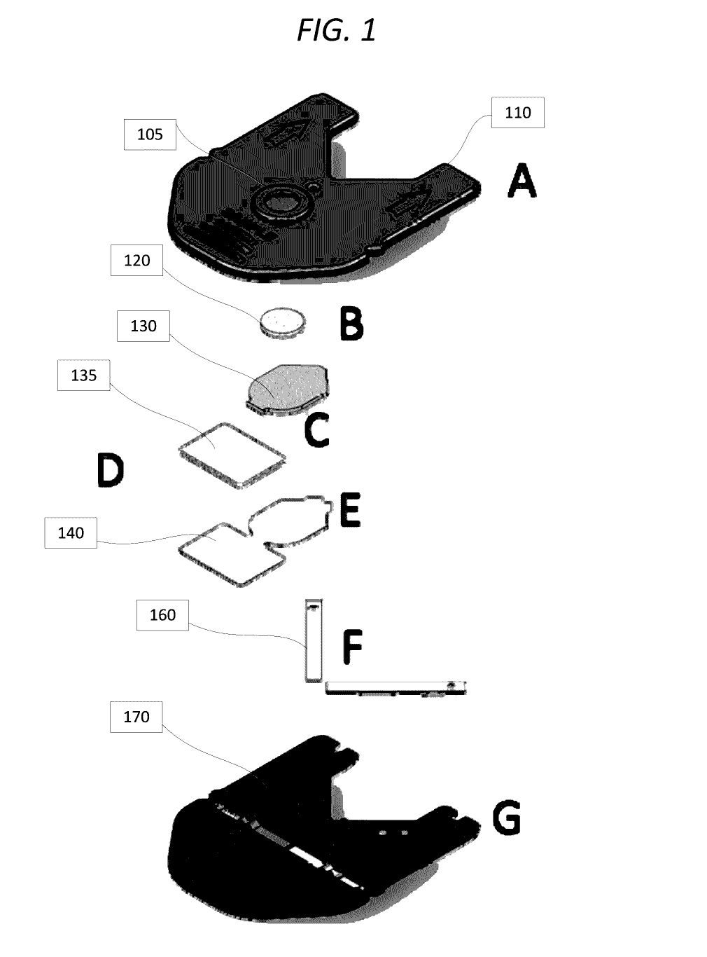

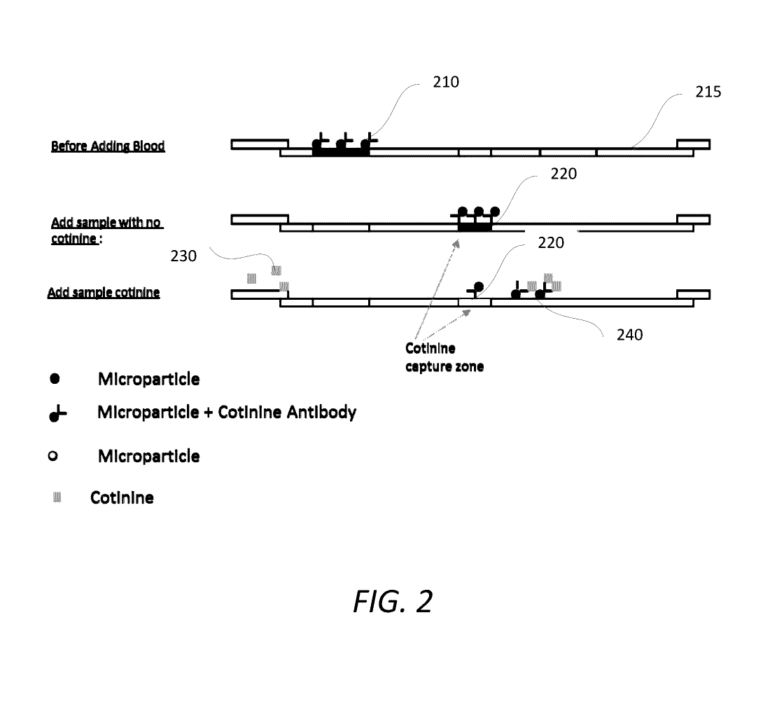

FIG. 1 shows one embodiment of a cartridge for use with a meter for reading a color change. In many embodiments, the sample is applied to sample pad 120 through the top opening 105 of the cartridge top 110 and quickly absorbed by sample pad 120. The treated blood sample then passes through the RBC separation membrane 130 where the RBCs are retained and the RBC-depleted sample progresses to the lateral wicking membrane 140. Various RBC depletion methodologies may be used, including filtering membranes and treated filtering membranes, for example. The sample then comes into contact with the lateral flow test strips 160, and an assay is performed as described by FIG. 2. The cartridge also includes a foam pad 135 for absorbing excess blood samples and a cartridge bottom 170.

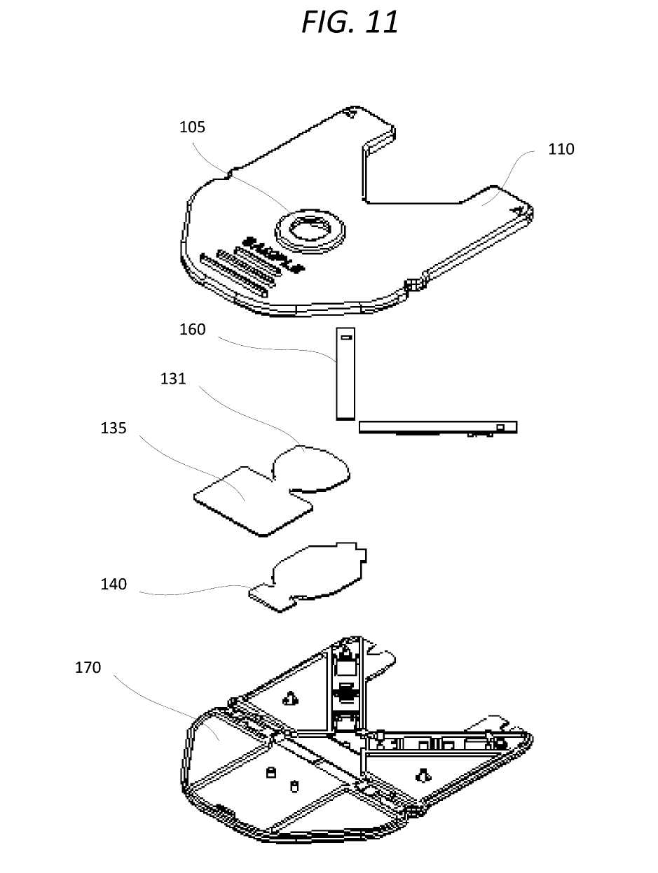

Various other configurations of the cartridge incorporating RBC separation are possible. One such example is shown in FIG. 11. In FIG. 11, a sample is applied to RBC separation membrane 131 through the top opening 105 of the cartridge top 110 and quickly absorbed. The blood sample then passes through the RBC separation membrane 131 where the RBCs are retained and the RBC-depleted sample progresses to the lateral wicking membrane 140. The sample then comes into contact with the lateral flow test strips 160, and an assay is performed as described by FIG. 2. The cartridge also includes a foam pad 135 for absorbing excess blood samples and a cartridge bottom 170. In the embodiment shown, foam pad 135 is interconnected with RBC separation membrane 131 such that excess blood may flow across the juncture between them. This narrow juncture ensures that the RBC separation membrane 131 becomes fully wetted, while allowing excess RBCs to transport to foam pad 135. Foam pad 135 may be made of the same material as RBC separation membrane 131 or an alternative material and simply interconnected with RBC separation membrane 131. Lateral wicking membrane 140 also includes a smaller absorption pad, separated similarly by a narrow junction.

In some embodiments, the lateral flow test strip portion includes two test strips for error checking and consistency purposes. The assay format may be a lateral-flow, a competitive-inhibition system where an antibody-coated particle is captured on a defined zone of antigen-mimicking conjugate on the lateral flow strip. Free antigen in the sample competes for antibody binding sites, preventing particle capture on the test zone, with low antigen concentrations resulting in the most capture and high concentrations resulting in less particle capture. The particles are dyed blue in the current embodiment, but any particle capable of producing transduction of a single indicator (i.e., optical, electrochemical, electromagnetic, etc.) can be used to quantify the amount of particle capture in the zone. In addition, the antigen/antibody placement can be reversed with the antigen mimicking conjugate placed on the particle and the antibody adhered to the capture zone on the lateral flow strip. FIG. 2 shows one embodiment of a schematic for competitive-inhibition, particle-capture immunoassay.

As can be seen in FIG. 2, before adding blood to the lateral flow test strip, microparticles with cotinine antibodies 210 are deposited in lateral flow test strip 215. The microparticles are dyed blue in this example, such that they may be detected by an optical meter. After a sample is added, if there is no cotinine in the sample, then no material bonds to the microparticles with cotinine antibodies 210 until the microparticles with cotinine antibodies 210 laterally flow to the cotinine capture zone 220. This zone is designed to bond with the microparticles with cotinine antibodies 210. If there is cotinine 230 in the sample, then, when the sample reaches the microparticles with cotinine antibodies 210, the cotinine 230 will bond with the microparticles with cotinine antibodies 210. In such a scenario, the microparticles with cotinine antibodies 210 with bonded cotinine 240 will not be captured in the cotinine capture zone 220 and will flow past it.

In some embodiments of the assay system, cartridges have demonstrated detection limits of ˜10 ng/mL and a potential dynamic range from 10 ng/mL to 600 ng/mL. The exact assay range can be optimized for sensitivity or large dynamic range depending on the conjugate and antibody loadings.

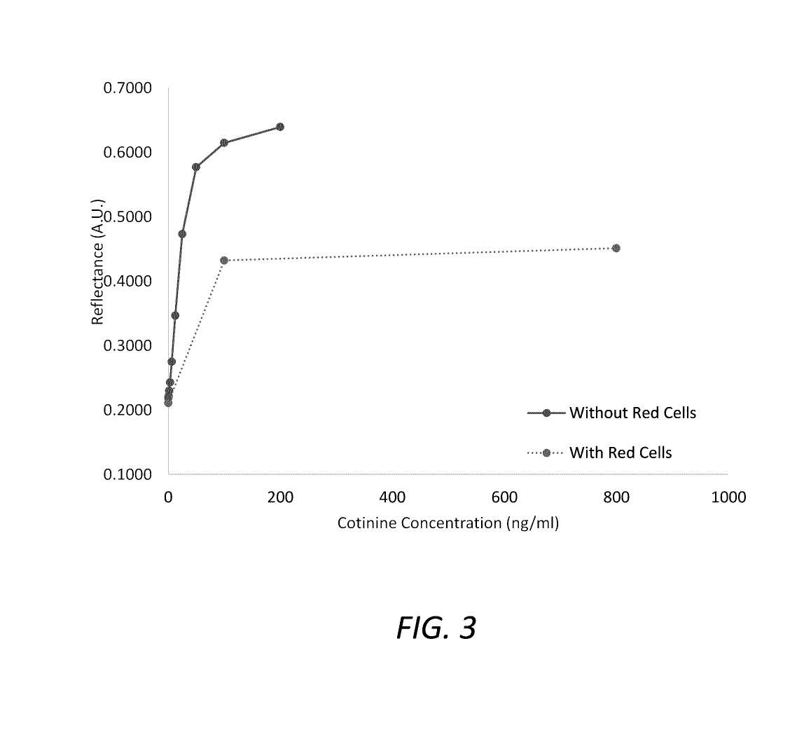

FIG. 3 demonstrates the effect of the included RBC separation step. As can be seen, whole blood and the inclusion of RBCs in the lateral flow sample cause a higher concentration of cotinine to be measured. The same is true for lysed RBC; therefore, the destruction of the cells with a lysing agent does not solve the hematocrit basis affecting the cotinine measurement.

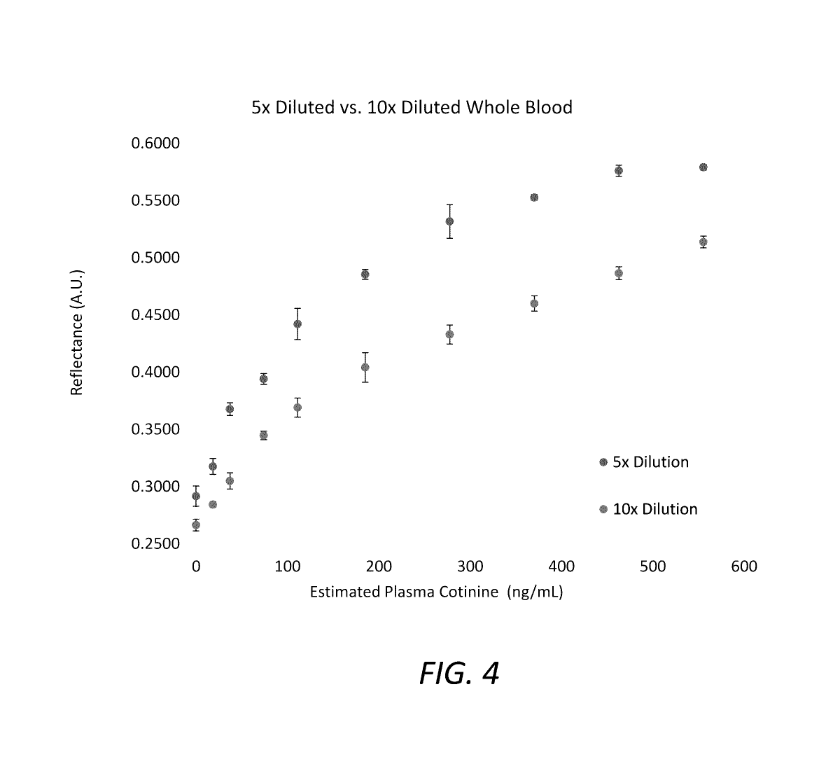

FIG. 4 demonstrates the effect of interference of RBCs on the reflectance measurement. Due to the effect of RBCs on the reflectance measured, in usage, it cannot be determined whether the reflectance reading is a result of cotinine in the sample or RBCs. One solution to this issue is to remove the RBCs using a vertical flow system. Another is to correct the measured reflectance based on the RBCs that an average individual has. Since the average RBCs for individuals may vary dramatically, the preference is to remove the RBCs, since the estimation method may significantly affect the accuracy of the system.

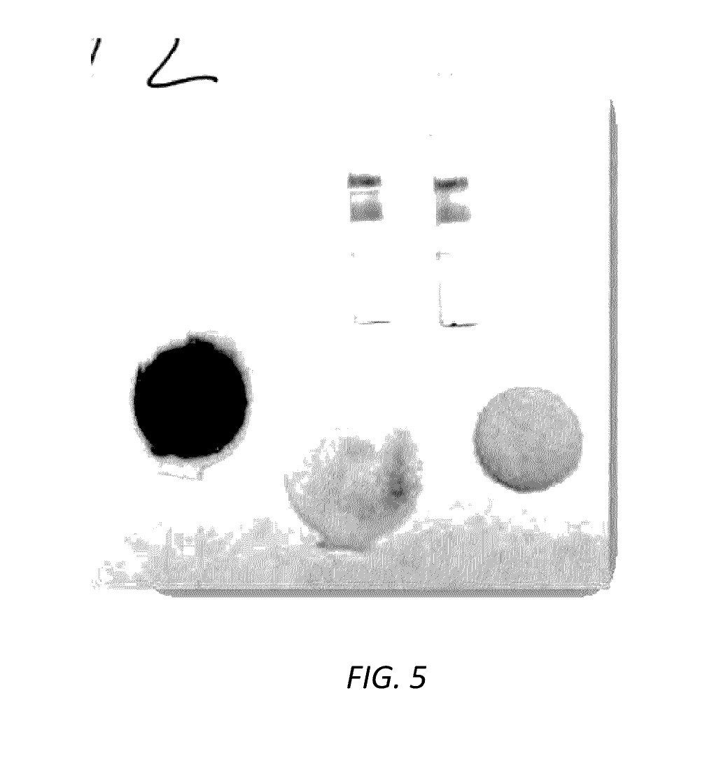

FIG. 5 shows a standard curve performed on prototype cartridges that included RBC separation system demonstration detection limits of ˜25 ng/mL. FIG. 5 shows the ability of the strips to separate the RBCs and the pristine nature of the reaction zone membranes relative to that in FIG. 3. This hybrid lateral-vertical flow system has advantages for all types of whole blood point-of-care assays where removal of blood cells prior to lysing is paramount.

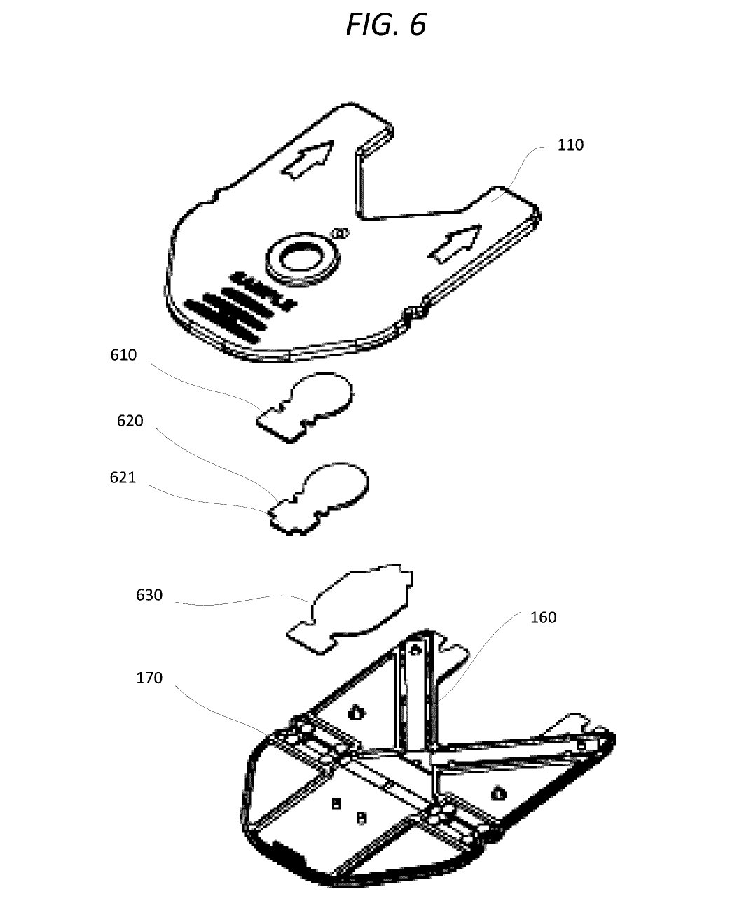

FIG. 6 shows an embodiment for a cartridge for detecting cotinine. Cartridge top 110 and cartridge bottom 170 enclose a stack of membranes and lateral flow strips 160. In this embodiment, a sample pad 610 receives a blood sample. The sample pad 610 absorbs the sample and transfers it to separation layer 620. Separation layer 620 is a physical separation layer for separating RBCs. The separation layer 620 may include a notch 621 as shown. In some configurations, notch 621 may serve to manage the sample size that reaches the layer below. Excess blood may be wicked towards this notch 621 and allowed to flow into an open area of the cartridge. Additionally, lateral wicking membrane 630 provides wicking to lateral flow test strips 160. The pore size of separation layer 620 and the other layers in combination may slow and filter the movement of RBCs to the lateral flow test strips 160. This is important, since either lysed or non-lysed RBCs can affect the color change, leading to an inaccurate test. Membranes may be composed of a variety of materials including glass, plastic, cellulous, and other materials, and may be woven or unwoven. In some embodiments, the separation layer is an asymmetric glass membrane having gradually narrowing pore apertures.

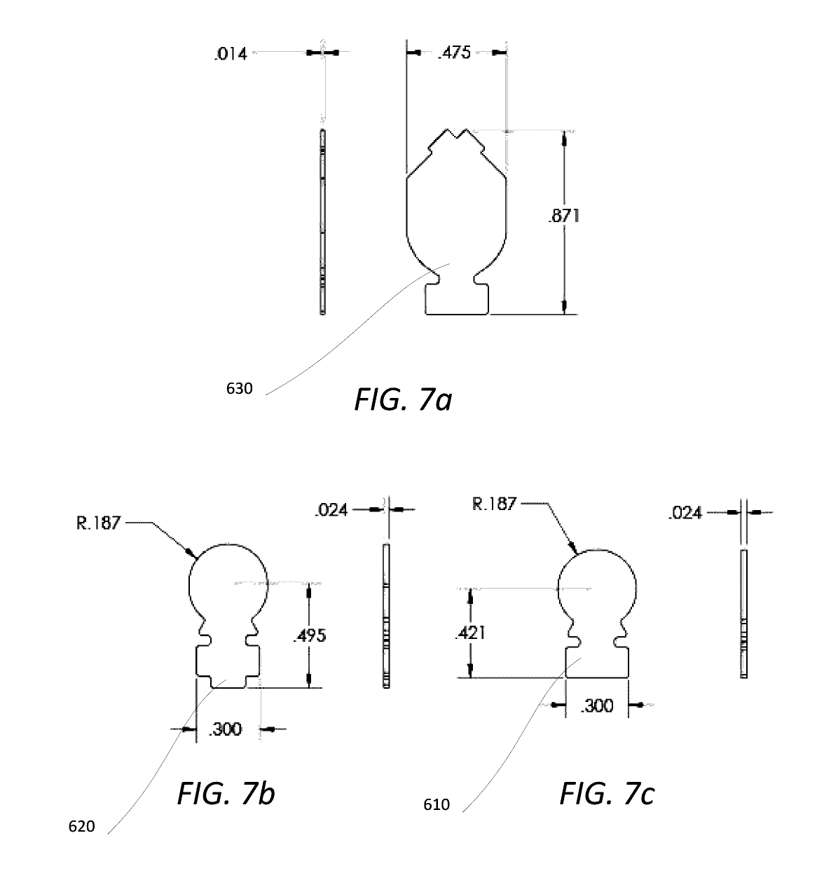

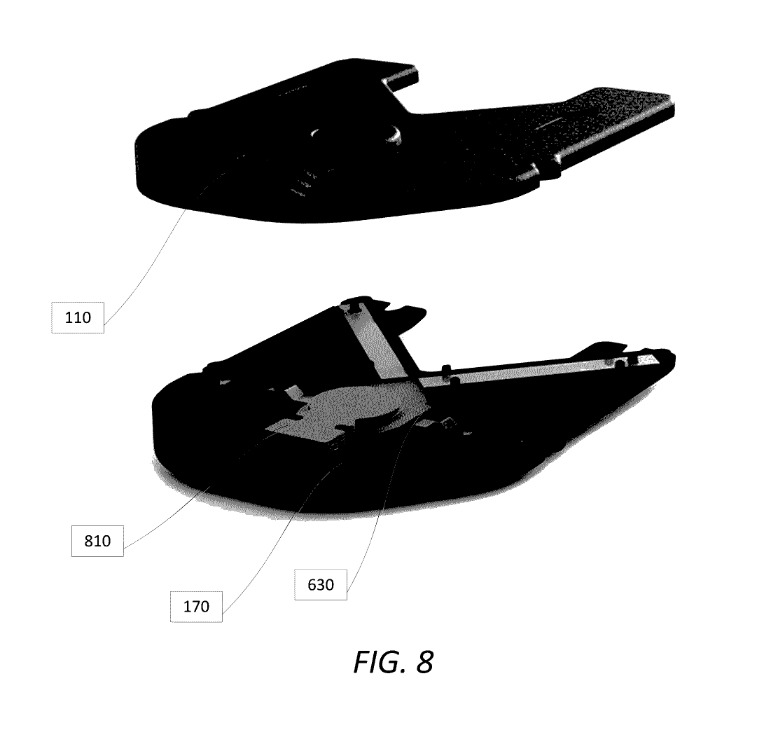

FIG. 7a shows a detailed view of one layer of the cartridge of FIG. 6. The lateral wicking membrane 630 provides for flow and contact with the lateral flow test strips 160. The dimensions of the membrane are shown in inches. FIG. 7b shows a detailed view of one layer 620 of the cartridge of FIG. 6. In some embodiments, separation layer 620 may be bound glass fiber. In some embodiments, it is MF1 22 mm×50 m available from GE Healthcare. The dimensions of the membrane are shown in inches. FIG. 7c shows a detailed view of one layer 610 of the cartridge of FIG. 6. In some embodiments, sample pad 610 is POR-41210, 0.024″ Polyethylene, 75-115 Microns 12″ Wide Rolls. The dimensions of the membrane are shown in inches. FIG. 8 shows a perspective view of the cartridge of FIG. 6. In FIG. 8, the alignment of stack 810 is shown. Stack 810 includes sample pad 610 and separation layer 620 and sits on top of lateral wicking membrane 630.

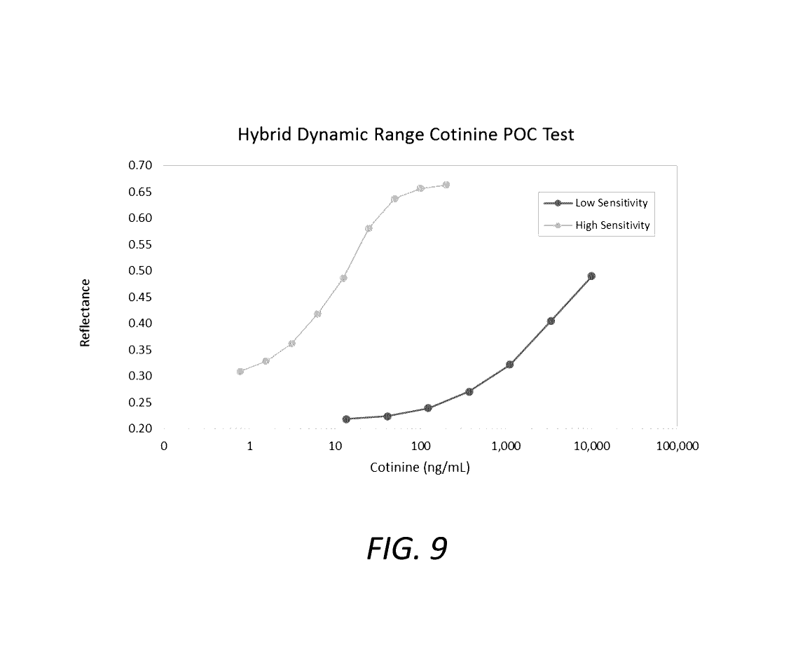

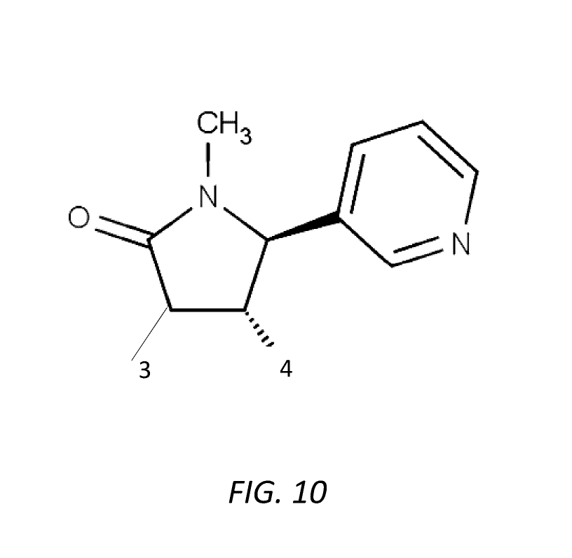

FIG. 9 shows one embodiment of a graph for the range for cotinine 3 and cotinine 4. Cotinine, as shown in FIG. 10, has two binding sites for protein; the 3^{rd} position carbon (cotinine 3) and the 4^{th} position carbon (cotinine 4). In the graph shown, a polyclonal antibody is used to bind to cotinine 4 and produce high sensitivity at lower levels. This is represented by the high sensitivity graph. Additionally, a monoclonal antibody is used to bind to cotinine 3 and produce additional detection sensitivity at higher ranges. The cotinine-specific antibodies may be deployed in microparticles with cotinine antibodies 210 as shown in FIG. 2. As shown in FIG. 1, there are two lateral flow test strips 160. In this case, different antibodies may be deployed for each lateral flow test strip. The meter then may read both test strips. If, in the test strip using the polyclonal antibody for cotinine 4 the maximum color, reflectivity, or other indicator is read by the test strip, then it is likely that the amount of cotinine in the sample has exceeded the range for the higher sensitivity but not the lower range lateral flow test strip. This scenario may occur when all of the microparticles with cotinine antibodies have bound with cotinine, resulting in no capture at cotinine capture zone 220. In such a scenario, the lateral flow test strip utilizing a monoclonal antibody to bind to cotinine 3 may be read. This lateral flow test strip provides for a higher range of readings. Additionally, in the range of approximately 10 ng/mL-100 ng/mL, the detection range of the lateral strips will overlap, therefore allowing for an accuracy cross check of readings detected in either lateral flow test strip.

While specific embodiments have been described in detail in the foregoing detailed description and illustrated in the accompanying drawings, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof.

What is claimed as new and desired to be protected by Letters Patent of the United States is:

1. A system for determining a level of cotinine in a sample, comprising:
a test strip system configured to receive a sample, the test strip system including a first lateral flow test strip and a second lateral flow test strip, the first and second lateral flow test strips each having an overlapping but non-identical range for cotinine; and
a meter configured to receive the test strip, wherein the meter is configured to read the test strip and detect a level of cotinine.

2. The system of claim 1, wherein the first and second lateral flow test strips each include microparticles combined with a cotinine antibody.

3. The system of claim 2, wherein the first and second lateral flow test strips each include compounds to bind with the microparticles combined with a cotinine antibody.

4. The system of claim 3, wherein for the first test strip the microparticles combined with a cotinine antibody include an antibody for cotinine 3.

5. The system of claim 3, wherein for the second test strip the microparticles combined with a cotinine antibody include an antibody for cotinine 4.

6. The system of claim 4, wherein for the second test strip the microparticles combined with a cotinine antibody include an antibody for cotinine 4, and the combination of the first and second test strips results in a larger testing range for cotinine using the test strip system.

7. The system of claim 6, wherein the antibody for cotinine 3 is monoclonal.

8. The system of claim 6, wherein the antibody for cotinine 4 is polyclonal.

9. The system of claim 6, wherein the test strip system includes a red blood cell separation membrane.

10. The system of claim 9, wherein the red blood cell separation membrane is a vertical flow membrane.

11. The system of claim 10, wherein the test strip system includes a sample pad oriented in line with an opening in a cartridge, the cartridge holding the sample pad, the red blood cell separation membrane, and the first and second lateral flow test strips.

12. The system of claim 10, wherein the test strip system includes a wicking membrane in a cartridge, and the cartridge holding the red blood cell separation membrane and the wicking membrane forms a stack of membranes in that order, the stack of membranes being approximately in vertical alignment with the opening, and the wicking membrane oriented in contact with the first and second lateral flow test strips in order to provide sample to the lateral flow test strips.

13. A system for determining a level of cotinine in a sample, comprising:
a test strip system configured to receive a sample; and
a meter configured to receive the test strip, wherein the meter is configured to read the test strip and detect a level of cotinine.

14. The system of claim 13, wherein the test strip system includes a red blood cell separation membrane.

15. The system of claim 14, wherein the test strip system includes a lateral flow test strip.

16. The system of claim 15, wherein the test strip system includes a red blood cell separation membrane.

17. The system of claim 16, wherein the red blood cell separation membrane is a vertical flow membrane.

18. The system of claim 17, wherein the test strip system includes a wicking membrane in the cartridge.

19. The system of claim 18, wherein a cartridge holds the red blood cell separation membrane and the wicking membrane and forms a stack of membranes in that order, the stack of membranes being approximately in vertical alignment with the opening, the wicking membrane oriented in contact with the lateral flow test strip in order to provide sample to the lateral flow test strip.

20. The system of claim 19, wherein the lateral flow test strip includes microparticles combined with a cotinine antibody.

21. The system of claim 20, wherein the test strip includes a first test site, the first test site including compounds to bind with the microparticles combined with a cotinine antibody.

22. The system of claim 21, wherein the microparticles are fluorescent.

23. The system of claim 21, wherein the microparticles have reflective properties.

24. The system of claim 21, wherein the microparticles have properties that provide for the absorption of light.

25. The system of claim 24, wherein the meter measures a level of absorption at the first test site to determine the level of cotinine.

26. The system of claim 23, wherein the meter measures a level of reflection at the first test site to determine the level of cotinine.

27. A method of determining a level of cotinine in a sample comprising:
providing a test strip system configured to receive a sample wherein the test strip system includes microparticles combined with a cotinine antibody;
providing a meter configured to receive the test strip wherein the meter is configured to read the test strip and detect a level of cotinine;
placing a sample on the test strip;
laterally flowing the sample of the test strip; and
reading the test strip with the meter.

28. The method of claim 27, wherein the test strip system includes:
a sample pad; and
a cartridge, the sample pad oriented in line with an opening in a cartridge, the cartridge holding the sample pad, the red blood cell separation membrane, and the lateral flow test strip.

29. The method of claim 28, wherein the test strip system includes a wicking membrane in the cartridge.

30. The method of claim 29, wherein the cartridge holding the sample pad, the red blood cell separation membrane, and the wicking membrane forms a stack of membranes in that order, the stack of membranes being approximately in vertical alignment with the opening, the wicking membrane oriented in contact with the lateral flow test strip in order to provide sample to the lateral flow test strip.

31. The method of claim 27, further comprising
binding at least a portion of cotinine with microparticles combined with the cotinine antibody; and
binding at least a portion of the microparticles combined with the cotinine antibody to a first test site;
wherein the reading of the test strip includes detecting at the first test site to determine the level of cotinine.
